SEQUENCE LISTING (19 sequences)

SEQ ID NO: 1 - protein, 10 aa, Homo sapiens
PMKTMKGPFG

SEQ ID NO: 2 - protein, 11 aa, Homo sapiens
PMKTMKGPFGG

SEQ ID NO: 3 - protein, 9 aa, Homo sapiens
PMKTMKGPF

SEQ ID NO: 4 - protein, 10 aa, synthetic construct
  MOD_RES 1: note = Biotinylated via an optional linker
PMKTMKGPFG

SEQ ID NO: 5 - protein, 10 aa, synthetic construct
VPKDLPPDTT

SEQ ID NO: 6 - protein, 10 aa, synthetic construct
  MOD_RES 1: note = Biotinylated
VPKDLPPDTT

SEQ ID NO: 7 - protein, 13 aa, synthetic construct
  MOD_RES 1: note = Bound to Keyhole Limpet Hemocyanin
CGGPMKTMKGPFG

SEQ ID NO: 8 - protein, 16 aa, Mus musculus
RSSQSIVHNNGNTYLE

SEQ ID NO: 9 - protein, 7 aa, Mus musculus
KVSNRFS

SEQ ID NO: 10 - protein, 9 aa, Mus musculus
FQGSHVPRT

SEQ ID NO: 11 - protein, 5 aa, Mus musculus
DYYIH

SEQ ID NO: 12 - protein, 17 aa, Mus musculus
WIDHDNGDTEYDPKFQG

SEQ ID NO: 13 - protein, 14 aa, Mus musculus
KGPRYGYEEDWFAY

SEQ ID NO: 14 - protein, 79 aa, Mus musculus
RSSQSIVHNNGNTYLEWFLQKPGQSPKLLIYKVSNRFSGVPDRFSGSGSGTDFTLRISRVEADDLGVYYCFQGSHVPRT

SEQ ID NO: 15 - protein, 82 aa, Mus musculus
DYYIHWVKQRPEQGLEWIGWIDHDNGDTEYDPKFQGKATLTADTSSNTAYLQLSSLTSEDTAVYYCNAKGPRYGYEEDWFAY

SEQ ID NO: 16 - protein, 112 aa, Mus musculus
DVLMTQTPLSLPVSLGDQASISCRSSQSIVHNNGNTYLEWFLQKPGQSPKLLIYKVSNRFSGVPDRFSGSGSGTDFTLRISRVEADDLGVYYCFQGSHVPRTFGGGTKLEIK

SEQ ID NO: 17 - protein, 123 aa, Mus musculus
EVQLQQSGAELVRSGASVKLSCTASGFNIKDYYIHWVKQRPEQGLEWIGWIDHDNGDTEYDPKFQGKATLTADTSSNTAYLQLSSLTSEDTAVYYCNAKGPRYGYEEDWFAYWGQGTLVTVST

SEQ ID NO: 18 - protein, 459 aa, Mus musculus
EVQLQQSGAELVRSGASVKLSCTASGFNIKDYYIHWVKQRPEQGLEWIGWIDHDNGDTEYDPKFQGKATLTADTSSNTAYLQLSSLTSEDTAVYYCNAKGPRYGYEEDWFAYWGQGTLVTVSTAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 19 - protein, 219 aa, Mus musculus
DVLMTQTPLSLPVSLGDQASISCRSSQSIVHNNGNTYLEWFLQKPGQSPKLLIYKVSNRFSGVPDRFSGSGSGTDFTLRISRVEADDLGVYYCFQGSHVPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2023/0280350 A1
(43) Pub. Date: Sep. 7, 2023

(54) Collagen Type XVI Assay

(71) Applicant: Nordic Bioscience A/S, Herlev (DK)

(72) Inventors: Christina Jensen, Kobenhavn N (DK); Joachim Hog Mortensen, Hundige Strand (DK); Nicholas Willumsen, Dyssegard (DK); Morten Asser Karsdal, Kobenhavn (DK)

(73) Assignee: Nordic Bioscience A/S, Herlev (DK)

(21) Appl. No.: 18/301,395

(22) Filed: Apr. 17, 2023

Related U.S. Application Data
(63) Continuation-in-part of application No. 16/757,572, filed on Apr. 20, 2020.

(30) Foreign Application Priority Data
Oct. 20, 2017 (EP) 1717301.4

(51) Int. Cl. G01N 33/574; C07K 16/18

(52) U.S. Cl. CPC G01N 33/57488; C07K 16/18; C07K 2317/565; G01N 2800/7028

(57) ABSTRACT

The present invention relates to a type XVI collagen assay and its use in evaluating diseases associated with type XVI collagen, in particular colorectal cancer and ulcerative colitis, and for identifying a subgroup of patients with Crohn’s disease that have (or are likely to develop) fibrostenotic strictures.

### CROSS-REFERENCE TO RELATED APPLICATIONS

This is a continuation-in-part application under 35 U.S.C. §120 of pending application U.S. Serial No. 16/757,572, filed Apr. 20, 2020, which is a national stage application under 35 U.S.C. §371 of International Application PCT/EP2018/078697, filed Oct. 19, 2018, now abandoned, which claimed priority to European Application No. 1717301.4, filed Oct. 20, 2017, now abandoned, the entireties of which are hereby incorporated by reference.

### REFERENCE TO AN ELECTRONIC SEQUENCE LISTING

The contents of the electronic sequence D7739CIPSEQ.xml with a size of 19 kb and created on Apr. 3, 2023 is hereby incorporated by reference in its entirety.

### BACKGROUND OF THE INVENTION

### Field of the Invention

The present invention relates to a type XVI collagen assay and its use in evaluating diseases associated with type XVI collagen, in particular colorectal cancer and ulcerative colitis, and for identifying a subgroup of patients with Crohn’s disease that have (or are likely to develop) fibrostenotic strictures.

### Description of the Related Art

The extracellular matrix (ECM) is a non-cellular component responsible for maintaining tissue architecture. Altered ECM remodelling is a significant part of the pathology of gastrointestinal (GI) disorders such as colorectal cancer (CRC) (1) and ulcerative colitis (UC) (2). An imbalance between ECM formation and degradation in the colon leads to an altered composition of the ECM thereby causing an abnormal tissue function. Elevated deposition of ECM proteins in the tumour microenvironment increase the stiffness of the ECM, which influences cellular functions such as cell proliferation, adhesion, migration and invasion (3,4). It has also become evident that inflammatory responses in the tumour microenvironment affect the ECM remodelling (5,6). Likewise, in UC, the ECM of the intestine is highly affected by chronic inflammation which leads to loss of tissue homeostasis and imbalanced collagen turnover (2,7-9). The chronic inflammation and the continuous turnover of epithelial cells contribute to development of dysplasia which may transform into CRC (10). Biomarkers reflecting this enhanced ECM remodelling may therefore be important to identify patients with disruption in tissue/ECM architecture responsible for development and progression of CRC and UC.

In the intestine, type XVI collagen (hereinafter col-16) is suggested to contribute to stabilization and maintenance of basement membranes, a specialized layer of ECM located beneath the epithelial and endothelial cell layers (11). Col-16 is a fibril-associated collagen with interrupted triple helices (FACITs), and expressed by epithelial cells and subepithelial myofibroblasts. These are localized subjacent to the basement membrane with a pronounced deposition of col-16 into the matrix of the epithelial crypts (11). Studies of skin show that col-16 is localized in the dermal-epidermal junction zone near basement membranes, which suggests that col-16 has an active role in anchoring microfibrils to basement membranes (12,13).

Col-16 interacts with α1β1 and α2β1 integrins and induces the recruitment of these integrins into focal adhesion plaques, which promotes integrin-mediated cell reactions, such as cell spreading and alterations in cell morphology (14,15). The binding of col-16 to integrins stimulates cell-matrix interactions, which it is postulated may induce an invasive phenotype in tumour cells. Interestingly, overexpression of col-16 has been shown to induce cell invasion and a proliferative cellular phenotype in oral squamous cell cancer (OSCC) (16,17). Col-16 is deposited at the basement membrane in normal oral epithelium while it seems to disappear from the basement membrane in tissues from OSCC patients (17). The loss of col-16 from the basement membrane zone in the development of OSCC may induce ECM remodelling and a disruption of the basement membrane that promotes tumour cell infiltration and a progression of disease. In glioblastomas, col-16 is involved in tumour cell adhesion and invasion as well as tumour specific remodelling of the ECM (18,19). Increased expression of col-16 has also been detected in intestinal subepithelial myofibroblasts isolated from inflamed Crohn’s disease tissue biopsies (11).

The inventors have now established a pathological link between col-16 and UC and CRC, and have also developed a method for identifying a subgroup of patients with Crohn’s disease that have (or are likely to develop) fibrostenotic strictures.

### SUMMARY OF THE INVENTION

Accordingly, in a first aspect the present invention relates to a method of detecting collagen type XVI or fragments thereof in a human biofluid sample, said method comprising:
a) obtaining a biofluid sample from a human patient; and
b) contacting the biofluid sample with a monoclonal antibody specifically reactive with a C-terminus biomarker having the C-terminus amino acid sequence PMKTMKGPFG (SEQ ID NO: 1) and detecting binding between the biomarker and the antibody.

The detection is preferably quantitative.

Preferably, the monoclonal antibody is raised against a synthetic peptide having the amino acid sequence PMKTMKGPFG (SEQ ID NO: 1). The synthetic peptide may be linked to a carrier protein such as, but not limited to, keyhole limpet hemocyanin (KLH).

In this regard “C-extended elongated version of said C-terminus amino acid sequence” means one or more amino acids extending beyond the C-terminus of the sequence PMKTMKGPFG-COOH (SEQ ID NO: 1). For example, if the C-terminal amino acid sequence PMKTMKGPFG-COOH (SEQ ID NO: 1) was elongated by a glycine residue then the corresponding “C-extended elongated version” would be PMKTMKGPFGG-COOH (SEQ ID NO: 2). Similarly, it is preferable that the antibody does not specifically recognise or bind a C-truncated shortened version of said C-terminus amino acid sequence. In this regard “C-truncated shortened version of said C-terminus amino acid sequence” means one or more amino acids removed from the C-terminus of the sequence PMKTMKGPFG-COOH (SEQ ID NO: 1). For example, if the C-terminal amino acid sequence PMKTMKGPFG-COOH (SEQ ID NO: 1) was shortened by one amino acid residue then the corresponding “C-truncated shortened version” would be PMKTMKGPF-COOH (SEQ ID NO: 3).

Monoclonal antibodies that specifically bind to the N-terminus amino acid sequence PMKTMKGPFG (SEQ ID No. 1) can be generated via any suitable techniques known in the art. For example, the monoclonal antibody may be raised against a synthetic peptide having the amino acid sequence PMKTMKGPFG (SEQ ID No. 1), such as for example by: immunizing a rodent (or other suitable mammal) with a synthetic peptide consisting of the sequence PMKTMKGPFG (SEQ ID No. 1), which optionally may linked to an immunogenic carrier protein (such as keyhole limpet hemocyanin), isolating and cloning a single antibody producing cell, and assaying the resulting monoclonal antibodies to ensure that they have the desired specificity. An exemplary protocol for producing a monoclonal antibody that that specifically bind to the N-terminus amino acid sequence PMKTMKGPFG (SEQ ID No. 1) is described infra.

Preferably, the monoclonal antibody or fragment thereof may preferably comprise one or more complementarity-determining regions (CDRs) selected from:

| | |
|---|---|
| CDR-L1: | RSSQSIVHNNGNTYLE (SEQ ID No. 8) |
| CDR-L2: | KVSNRFS (SEQ ID No. 9) and |
| CDR-L3: | FQGSHVPRT (SEQ ID No. 10) |
| CDR-H1: | DYYIH (SEQ ID No. 11) |
| CDR-H2: | WIDHDNGDTEYDPKFQG (SEQ ID No. 12) and |
| CDR-H3: | KGPRYGYEEDWFAY (SEQ ID No. 13) |

Preferably the antibody or fragment thereof comprises at least 2, 3, 4, 5 or 6 of the above listed CDR sequences.

Preferably the monoclonal antibody or fragment thereof has a light chain variable region comprising the CDR sequences

| | |
|---|---|
| CDR-L1: | RSSQSIVHNNGNTYLE (SEQ ID No. 8) |
| CDR-L2: | KVSNRFS (SEQ ID No. 9) and |
| CDR-L3: | FQGSHVPRT (SEQ ID No. 10) |

Preferably the monoclonal antibody or fragment thereof has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

RSSQSIVHNNGNTYLE WFLQKPGQSPKLLIYKVSNRFS GVPDRFSGSGSG TDFTLRISRVEADDLGVYYCFQGSHVPRT (SEQ ID No. 14)

Preferably the monoclonal antibody or fragment thereof has a heavy chain variable region comprising the CDR sequences

| | |
|---|---|
| CDR-H1: | DYYIH (SEQ ID No. 11) |
| CDR-H2: | WIDHDNGDTEYDPKFQG (SEQ ID No. 12) and |
| CDR-H3: | KGPRYGYEEDWFAY (SEQ ID No. 13) |

Preferably the monoclonal antibody or fragment thereof has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

DYYIH WVKQRPEQGLEWIGWIDHDNGDTEYDPKFQG KATLTADTSSNTAY LQLSSLTSEDTAVYYCNAKGPRYGYEEDWFAY (SEQ ID No. 15)

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody or fragment thereof may comprise the light chain variable region sequence:

DVLMTQTPLSLPVSLGDQASISCRSSQSIVHNNGNTYLE WFLQKPGQSPK LLlYKVS NRFS GVPDRFSGSGSGTDFTLRISRVEADDLGVYYCFQGSHVP RT FGGGTKLEIK(SEQ ID No. 16)

(CDRs bold and underlined; Framework sequences in italics) and/or the heavy chain variable region sequence:

EVQLQQSGAELVRSGASVKLSCTASGFNIKDYYIH WVKQRPEQGLEWIGW IDHDN GDTEYDPKFQG KATLTAOTSSNTAYLQLSSLTSEOTAVYYCNAKG PRYGYEEDWFAY WGQGTLVTVST (SEQ ID No. 17)

(CDRs bold and underlined; Framework sequences in italics)

Through the use of various statistical analyses it has been found that a measured amount of binding between the monoclonal antibody (described above) and the C-terminus biomarker of 1.0 ng/mL or greater is associated with a high likelihood of ulcerative colitis or colorectal cancer. In that regard, it was found that of the total population screened (inclusive of healthy subjects, UC patients and CRC patients), at least 90% of the subjects in that population that had a said C-terminus biomarker level of ≥1.0 ng/mL suffered from UC or CRC. As such, by setting a cutoff value of 1.0 ng/mL it is possible to utilise the method of the invention to predict the likelihood of ulcerative colitis or colorectal cancer with a high level of confidence. Or, in other words, applying the statistical cutoff value to the method of the invention is particularly advantageous as it results in a standalone predictive assay; i.e. it removes the need for any direct comparisons with healthy individuals and/or patients with known disease severity in order to arrive at a diagnostic conclusion. This may also be particularly advantageous when utilising the assay to evaluate patients that already have medical signs or symptoms that are generally indicative of colorectal cancer or ulcerative colitis (e.g. as determined by a physical examination and/or consultation with a medical professional) as it may act as a quick and definitive tool for corroborating the initial prognosis and thus potentially remove the need for more invasive procedures, such as an endoscopy, and expedite the commencement of a suitable treatment regimen. In the particular case of colorectal cancer an expedited conclusive diagnosis may result in the disease being detected and treated at an earlier stage, which may in turn improve overall chances of survival. The statistical cutoff value may therefore be used for assessing the risk of a patient having or developing UC or CRC.

Furthermore, where the patient has Crohn’s disease, it has been found that a measured amount of binding between the monoclonal antibody (described above) and the C-terminus biomarker of 1.7 ng/mL (statistical cutoff value) or greater is associated with a high likelihood of said patient having, or being likely to develop, fibrostenotic strictures. This is advantageous for much the same reasons as set out above.

Thus, in a second aspect, the present invention relates to an immunoassay method for diagnosing and/or monitoring and/or assessing the likelihood of colorectal cancer or ulcerative colitis in a patient, the method comprising contacting a biofluid sample obtained from said patient with an antibody reactive with collagen type XVI or fragments thereof, determining the amount of binding between said antibody and collagen type XVI or fragments thereof, and correlating said amount of binding with values associated with normal healthy subjects and/or values associated with known disease severity and/or values obtained from said patient at a previous time point and/or a predetermined statistical cutoff value.

Specifically, the method may be used to monitor the progression of UC or CRC in a patient, and/or the effects of therapy on a patient suffering from UC or CRC. For example, a first value may be obtained at a first time point prior to the commencement of therapy and a second value may be obtained at a second later time point after the commencement of therapy. A reduction in the amount of binding as measured by the method from the first time point to the second time point would be indicative of an improvement of the patient’s condition, hence demonstrating the patient is responding to the therapy. Conversely, an increase in the amount of binding as measured by the method from the first time point to the second time point would be indicative of a deterioration of the patient’s condition, hence demonstrating that the patient is not responding to the therapy. Accordingly, the method may be used to monitor and/or evaluate the efficacy of a novel therapeutic, such as, but not limited to, a new drug or antibody therapy.

Similarly, where the patient has Crohn’s disease, the present invention relates to an immunoassay method for diagnosing the presence of fibrostenotic strictures or assessing the likelihood of development of fibrostenotic strictures in the patient with Crohn’s disease, the method comprising contacting a biofluid sample obtained from said patient with an antibody specifically reactive with a C-terminus biomarker having the C-terminus amino acid sequence PMKTMKGPFG (SEQ ID NO: 1), and determining the amount of binding between said antibody and said biomarker, wherein a determined amount of binding of 1.7 ng/mL or greater is indicative of the presence of or likelihood of development of fibrostenotic strictures in said patient. Again, this method is advantageous for much the same reasons as set out above (monitoring disease progression, effects of therapy, etc.).

The detection is preferably quantitative. The fragments are preferably C-terminus fragments of Collagen type XVI.

Preferably, the antibody is specifically reactive with the C-terminus biomarker having the amino acid sequence PMKTMKGPFG (SEQ ID NO: 1). More preferably, the antibody is a monoclonal antibody. Preferably still, the antibody does not specifically recognise or bind a C-extended elongated version of said C-terminus amino acid sequence or a C-truncated shortened version of said C-terminus amino acid sequence.

For the reasons set out above, the immunoassay method of the second aspect may utilise the statistical cutoff value of 1.0 ng/mL of the C-terminus biomarker to determine the likelihood of UC or CRC.

In any of the methods of the invention described herein, the biofluid sample may be, but is not limited to, blood, urine, synovial fluid, serum or plasma.

In any of the methods of the invention described herein, the method of immunoassay may be, but is not limited to, a competition assay or a sandwich assay. The method of immunoassay may be, but is not limited to, a radioimmunoassay or an enzyme-linked immunosorbent assay.

If the patient is determined to have colorectal cancer as the amount of binding detected is determinative of the presence of colorectal cancer, then the method may further comprise the step of treating the patient. This may involve administering to the patient suitable treatment for colorectal cancer. Treatments for colorectal cancer include surgery, immunotherapy, cryotherapy, radiofrequency ablation, selective internal radiation therapy (SIRT), stereotactic ablative radiotherapy (SABR), photodynamic therapy, radiotherapy, chemotherapy including capecitabine, fluorouracil (5FU), folinic acid (leucovorin or calcium folinate) fluorouracil and oxaliplatin (FOLFOX), irinotecan (Campto), or oxaliplatin and capecitabine; chemoradiotherapy, targeted therapies including cetuximab, panitumumab, bevacizumab, afibercept, ramucirumab, regorafenib alone or in combination with chemotherapy.

If the patient is determined to have ulcerative colitis or Crohn’s disease as the amount of binding detected is determinative of the presence of ulcerative colitis or Crohn’s disease, then the method may further comprise the step of treating the patient. This may involve administering to the patient suitable treatment for ulcerative colitis or Crohn’s disease. Treatments for ulcerative colitis or Crohn’s disease include aminosalicylates, Corticosteroids, such as prednisolone and budesonide, Immunosuppressants such as azathioprine, mercaptopurine, tacrolimus and azathioprine, ciclosporin, JAK inhibitors such as tofacitinib and filgotinib, Biologic medicines such as infliximab, adalimumab, ustekinumab and vedolizumab and surgery.

In a third aspect the present invention relates to an assay kit comprising a monoclonal antibody specifically reactive with a C-terminus biomarker having the amino acid sequence PMKTMKGPFG (SEQ ID NO: 1), and at least one of:
a streptavidin coated well plate
a biotinylated peptide Biotin-L-PMKTMKGPFG (SEQ ID NO: 4), wherein L is an optional linker
a secondary antibody for use in a sandwich immunoassay
a calibrator peptide comprising the sequence PMKTMKGPFG-COOH (SEQ ID NO: 1)
an antibody biotinylation kit
an antibody HRP labeling kit
an antibody radiolabeling kit
an assay visualization kit

The kit may be used for diagnosing or predicting the risk of ulcerative colitis or colorectal cancer, or for identifying patients with Crohn’s disease that have or are likely to develop fibrostenotic strictures disease phenotype, preferably in conjunction with any of the methods described above.

### BRIEF DESCRIPTION OF THE FIGURES

FIG. 1: Specificity of the PRO-C16 assay (also referred to as “C16-C” in the Figures). %B/B0: B equals the OD at x nM peptide and B0 equals the OD at 0 nM peptide.

FIGS. 2A-2C: Serum PRO-C16 levels in patients with colorectal cancer (CRC) and ulcerative colitis (UC) compared to healthy controls; FIG. 2A: PRO-C16 levels in serum from controls (n=50), CRC (n=50) and UC patients (n=39). Levels below lower limit of measurement range (LLMR) are adjusted to LLMR. Results are presented as Tukey box plots. The boxes represent the 25th and 75th percentiles with a median. The whiskers represent the lowest and highest value, except outliers (+), which are higher than 1.5 times the 75th percentile. Groups were compared using Kruskal Wallis test. Asterisks indicate the following: **, p<0.01 and ****, p<0.0001; FIG. 2B: Levels of PRO-C16 in serum from CRC patients, UC patients and controls divided by quartiles (Q). The number of controls, CRC and UC patients in each group are illustrated. The cutoff value (1.0 ng/mL) obtained from a ROC curve are illustrated by a dotted line; FIG. 2C: PRO-C16 levels were compared in serum from CRC patients at baseline and three months after tumor resections (follow up). Statistical significant difference was determined using the paired Wilcoxon test. p>0.9999

FIG. 3. Evaluation of PRO-C16 in serum from colorectal cancer (CRC) patients according to tumor stage. Levels of PRO-C16 in serum from CRC patients at baseline divided into stage of disease with the median illustrated by a horizontal line. Groups were compared using Kruskal-Wallis test.

FIG. 4. Evaluation of PRO-C16 in serum from patients with Crohn’s disease. B1: CD patients with luminal disease; B2: CD patients with fibrostenotic strictures disease phenotype; B3: CD patients with fistulizing disease phenotype.

### DETAILED DESCRIPTION OF THE INVENTION

### Definitions

As used herein the term “C-terminus” refers to the extremity of a polypeptide, i.e. at the C-terminal end of the polypeptide, and is not to be construed as meaning in the general direction thereof.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, the monoclonal antibody may comprise any constant region known in the art. Human constant light chains are classified as kappa and lambda light chains. Heavy constant chains are classified as mu, delta, gamma, alpha, or epsilon, and define the antibody’s isotype as IgM, IgD, IgG, IgA, and IgE, respectively. The IgG isotype has several subclasses, including, but not limited to IgGI, IgG2, IgG3, and IgG4. The monoclonal antibody may preferably be of the IgG isotype, including any one of IgGI, IgG2, IgG3 or IgG4.

The CDR of an antibody can be determined using methods known in the art such as that described by Kabat et al¹⁹. Antibodies can be generated from B cell clones as described in the examples. The isotype of the antibody can be determined by ELISA specific for human IgM, IgG or IgA isotype, or human IgG1, IgG2, IgG3 or IgG4 subclasses. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

As used herein the term “ELISA” (enzyme-linked immunosorbent assay) refers to an immunoassay in which the target peptide present in a sample (if any) is detected using antibodies linked to an enzyme, such as horseradish peroxidase or alkaline phosphatase. The activity of the enzyme is then assessed by incubation with a substrate generating a measurable product. The presence and/or amount of target peptide in a sample can thereby be detected and/or quantified. ELISA is a technique known to those skilled in the art.

As used herein the term, the term “competitive ELISA” refers to a competitive enzyme-linked immunosorbent assay. In a “competitive ELISA” the target peptide present in a sample (if any) competes with known amount of target of peptide (which for example is bound to a fixed substrate or is labelled) for to binding an antibody, and is a technique known to the person skilled in the art.

As used herein the term “sandwich immunoassay” refers to the use of at least two antibodies for the detection of an antigen in a sample, and is a technique known to the person skilled in the art.

As used herein the term “amount of binding” refers to the quantification of binding between antibody and biomarker, which said quantification is determined by comparing the measured values of biomarker in the biofluid samples against a calibration curve, wherein the calibration curve is produced using standard samples of known concentration of the biomarker. In the specific assay disclosed herein which measures in biofluids the C-terminus biomarker having the C-terminus amino acid sequence PMKTMKGPFG (SEQ ID NO: 1), the calibration curve is produced using standard samples of known concentration of the calibration peptide PMKTMKGPFG (SEQ ID NO: 1). The values measured in the biofluid samples are compared to the calibration curve to determine the actual quantity of biomarker in the sample. The present invention utilises spectrophotometric analysis to both produce the standard curve and measure the amount of binding in the biofluid samples; in the Examples set out below the method utilises HRP and TMB to produce a measurable colour intensity which is proportional to the amount of binding and which can be read by the spectrophotometer. Of course, any other suitable analytical method could also be used.

As used herein the “statistical cutoff value” means an amount of binding that is determined statistically to be indicative of a high likelihood of UC or CRC in a patient, or indicative of a Crohn’s disease (CD) patient with (or likely to develop) fibrostenotic strictures, in that a measured value of biomarker in a patient sample (preferably a serum sample) that is at or above the statistical cutoff value corresponds to at least an 80% probability, preferably at least an 85% probability, more preferably at least a 90% probability, and most preferably at least a 95% probability of the presence or likelihood of UC or CRC, or (for CD patients) the presence or likelihood of developing fibrostenotic strictures.

As used herein the term “values associated with normal healthy subjects and/or values associated with known disease severity” means standardised quantities of collagen type XVI determined by the method described supra for subjects considered to be healthy, i.e. without UC or CRC, and/or standardised quantities of collagen type XVI determined by the method described supra for subjects known to have UC or CRC of a known severity.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.

### Materials

All reagents used for the experiments were standard chemicals from Merck (Whitehouse station, NJ, USA) and Sigma Aldrich (St. Louis, MO, USA). The synthetic peptides used for antibody production and assay development were purchased from the Chinese Peptide Company (Beijing, China) (Table 1). Selection and overview of peptides

### Epitope Selection

The C-terminal of the α1 chain of col-16 was chosen as the target epitope and is herein referred to as “PRO-C16”. The PRO-C16 amino acid sequence 1595′-PMKTMKGPFG (SEQ ID NO: 1) located at the C-terminal was used to generate an antibody specific for the C-terminal of col-16. It was additionally used to design the selection peptide (PMKTMKGPFG; SEQ ID NO: 1) (Table 1). The sequence was BLASTed for homology to other human proteins and species using the NPS@: network protein sequence analysis with the Uniprot/Swiss-Prot database (20). The amino acid sequence is unique to human col-16. A biotinylated peptide (Biotin-K-PMKTMKGPFG) was used to coat the streptavidin-coated plates applied in the ELISA. An elongated peptide (PMKTMKGPFGG; SEQ ID NO: 2), a truncated peptide (PMKTMKGPF; SEQ ID NO: 3), a nonsense peptide (VPKDLPPDTT; SEQ ID NO: 5) and a nonsense biotinylated peptide (Biotin-VPKDLPPDTT; SEQ ID NO: 6) were included to test the specificity of the antibody.

TABLE 1

Synthetic peptides used for antibody production and assay development

| Peptide | Sequence | SEQ ID NO |
|---|---|---|
| Selection peptide | PMKTMKGPFG | 1 |
| Immunogenic peptide | KLH-CGG-PMKTMKGPFG | 7 |
| Biotinylated peptide | Biotin-KPMKTMKGPFG | 4 |
| Elongated peptide | PMKTMKGPFGG | 2 |
| Truncated peptide | PMKTMKGPF | 3 |
| Nonsense peptide | VPKDLPPDTT | 5 |
| Nonsense biotinylated peptide | Biotin-VPKDLPPDTT | 6 |

KLH, Keyhole Limpet hemocyanin

### Monoclonal Antibody Production and Clone Characterization

Generation of monoclonal antibodies was carried out as previously described (21). Briefly, four to six week old Balb/C mice were immunized subcutaneously with 200 µl emulsified antigen and 50 µg immunogenic peptide (Keyhole Limpet Hemocyanin (KLH)-CGG-PMKTMKGPFG; SEQ ID NO: 7) using Freund’s incomplete adjuvant (Sigma-Aldrich, St. Louis, MO, USA). The mice were immunized with two-week intervals until stable serum titer levels were reached. The mouse with the highest serum titer was selected for fusion, rested for one month, then immunized intravenously with 50 µg immunogenic peptide in 100 µl 0.9% NaCl solution. After three days, splenocytes were isolated for cell fusion. In brief, splenocytes were fused with SP2/0 myeloma cells to produce hybridoma cells, and then cloned in culture dishes using the semi-medium method (22). The clones were plated into 96-well microtiter plates and limited dilution was used to secure monoclonal growth. The supernatants were screened for reactivity against the selection peptide (PMKTMKGPFG; SEQ ID NO: 1) and native material (serum) in an indirect competitive ELISA using streptavidin-coated plates (Roche, Hvidovre, Denmark, cat. 11940279). The clones with the best reactivity were purified using protein-G-columns according to the manufacturer’s instructions (GE healthcare Life Sciences, Little Chalfont, Buckinghamshire, UK). Two monoclones were tested for their reactivity towards the selection peptide (PMKTMKGPFG; SEQ ID NO: 1) and not the elongated (PMKTMKGPFGG; SEQ ID NO: 2), truncated (PMKTMKGPF; SEQ ID NO: 3) or nonsense peptide (VPKDLPPDTT; SEQ ID NO: 5). One monoclone was chosen for assay development. Optimal incubation buffer, time, temperature and optimal ratio between the biotinylated peptide and antibody was determined.

The antibody generated was sequenced and the CDRs determined. Total RNA was isolated from the hybridoma cells following the technical manual of TRIzol® Reagent (Ambion, Cat. No. : 15596-026). Total RNA was then reverse-transcribed into cDNA using either isotype-specific anti-sense primers or universal primers following the technical manual of SMARTScribe Reverse transcriptase. Antibody fragments of heavy chain and light chain were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes. The consensus sequence was provided.

The sequence of the chains are as follows (CDRs in bold; Framework sequence in Italics; Constant region underlined): Heavy chain: Amino acid sequence (478aa) (Mouse IgG2b isotype)

EVQLQQSGAELVRSGASVKLSCTASGFNIKDYYIH WVKQRPEQGLEWIGW IDHDN GDTEYDPKFQG KATLTADTSSNTAYLQLSSLTSEDTAVYYCNAKG PRYGYEEDWFAY WGQGTLVTVSTAKTTPPSVYPLAPGCGDTTGSSVTLGC LVKGYFPESVTVTWNSGSLSSSVHTFPALLQSGLYTMSSSVTVPSSTWPS QTVTCSVAHPASSTTVDKKLEPSGPISTINPCPPCKECHKCPAPNLEGGP SVFIFPPNIKDVLMISLTPKVTCVVVDVSEDDPDVQISWFVNNVEVHTAQ TQTHREDYNSTIRVVSTLPIQHQDWMSGKEFKCKVNNKDLPSPIERTISK IKGLVRAPQVYILPPPAEQLSRKDVSLTCLVVGFNPGDISVEWTSNGHTE ENYKDTAPVLDSDGSYFIYSKLNMKTSKWEKTDSFSCNVRHEGLKNYYLK KTISRSPGK (SEQ ID. NO:18)

CDR-H1: DYYIH (SEQ ID No. 11)
CDR-H2: WIDHDNGDTEYDPKFQG (SEQ ID No. 12) and
CDR-H3: KGPRYGYEEDWFAY (SEQ ID No. 13)

Light chain: Amino acid sequence (238 aa) (mouse Kappa isotype)

DVLMTQTPLSLPVSLGDQASISCRSSQSIVHNNGNTYLE WFLQKPGQSPK LLIYKVSNRFS GVPDRFSGSGSGTDFTLRISRVEADDLGVYYCFQGSHVP RT FGGGTKLEIKRADAAPTVSIFPPSSEQLTSGGASVVCFLNNFYPKDIN VKWKIDGSERQNGVLNSWTDQDSKDSTYSMSSTLTLTKDEYERHNSYTCE ATHKTSTSPIVKSFNRNEC (SEQ ID.NO:19)

CDR-L1: RSSQSIVHNNGNTYLE (SEQ ID No. 8)
CDR-L2: KVSNRFS (SEQ ID No. 9) and
CDR-L3: FQGSHVPRT (SEQ ID No. 10)

### PRO-C16 ELISA Protocol

The PRO-C16 competitive ELISA procedure was as follows; a 96-well streptavidin-coated microtiter plate was coated with 100 µl of biotinylated peptide (Biotin-K-PMKTMKGPFG; SEQ ID NO: 4) dissolved in assay buffer (50 mM PBS-BTB, 4 g/l NaCl, pH 7.4) (final concentration of 3.1.0 ng/mL). The plate was incubated for 30 minutes at 20° C. with shaking (300 rpm) and then washed five times in washing buffer (20 mM TRIS, 50 mM NaCl, pH 7.2). A volume of 20 µl of sample/control/selection peptide (PMKTMKGPFG; SEQ ID NO: 1) was added followed by immediately addition of 100 µl of monoclonal antibody diluted in assay buffer (final concentration of 62.5 ng/ml). The plate was incubated for 1 hour at 20° C. with shaking followed by five washes in washing buffer. Then, 100 µl of goat anti-mouse HRP-conjugated IgG antibody (Thermo Scientific, Waltham, MA, USA; cat. #31437) diluted in assay buffer (final concentration of 130 ng/ml) was added to each well. The plate was incubated for 1 hour at 20° C. with shaking and subsequently washed five times in washing buffer. Next, 100 µl Tetramethylbenzidine (TMB, Kem-En-Tec Diagnostics, Taastrup, Denmark) was added and incubated for 15 minutes at 20° C. with shaking in the dark. To stop the reaction of TMB, 100 µl of 1% H₂SO₄ was added and the plate was analyzed in a VersaMax ELISA microplate reader at 450 nm with 650 nm as reference. A standard curve was plotted using a 4-parametric mathematical fit model and data were analyzed using the Softmax Pro v. 6.3 software.

### Technical Evaluation

Antibody specificity was calculated as percentage of signal inhibition of 2-fold diluted selection peptide (PMKTMKGPFG; SEQ ID NO: 1), elongated peptide (PMKTMKGPFGG; SEQ ID NO: 2), truncated peptide (PMKTMKGPF; SEQ ID NO: 3) or nonsense peptide (VPKDLPPDTT; SEQ ID NO: 5). Lower limit of measurement range (LLMR) and upper limit of measurement range (ULMR) were calculated based on the standard curve from 10 independent runs. A 2-fold dilution of healthy serum samples from humans (n=3) were used to determine linearity and calculated as percentage recovery of the undiluted sample. Ten independent runs of seven samples that covered the detection range (LLMR-ULMR) of the PRO-C16 assay were used to calculate the intra-and-inter-assay variation. The seven samples included three human serum samples and four samples with selection peptide spiked in assay buffer. The intra-assay variation was determined as the mean coefficient of variance (CV%) within plates, and the inter-assay variation was calculated as the mean CV% between plates. Accuracy was determined from three human serum samples spiked with two-fold dilutions of the selection peptide and calculated as percentage recovery of the expected concentration (serum and peptide combined). The analyte stability was determined for three healthy serum samples subjected to up to four freeze and thaw cycles. The freeze-thaw recovery was calculated with the first cycle as reference. Analyte stability was furthermore determined by incubation of three human serum samples at either 4° C. or 20° C. for 24 or 48 hours. Recovery was calculated with samples stored at -20° C. as reference. Interference was determined by adding low/high content of biotin (1.5/4.5 ng/ml), lipid (0.75/2.5 mg/ml) or hemoglobin (1.25/2.5 mg/ml) to serum samples of known concentrations and calculated as the percentage recovery of analyte in non-spiked serum.

### Patient Serum Samples

Serum samples from CRC patients were collected by medical staff at Bispebjerg hospital, Copenhagen, Denmark subsequent to informed consent and approval by the Ethical Committee of the Capital Region of Denmark (Copenhagen, Denmark; approval no. H-1-2014-048) in compliance with the Helsinki Declaration. Serum samples were collected before (baseline) and three months after tumour resections (follow up) from 50 and 23 patients, respectively. Tumour staging was evaluated according to the Union for International Cancer Control classification system.

Serum samples from UC patients (n=39) were obtained from Odense University Hospital (Odense, Denmark) after informed consent. Levels of PRO-C16 in the CRC and UC patients were compared to levels in commercially available control sera from healthy donors (n=50) (Valley BioMedical, Winchester, VA, USA) which according to manufacturer’s information all filed informed consent. Information associated with the included patients is shown in Table 2. According to Danish law, it is not required to get additional ethical approval when measuring biochemical markers in previously collected samples.

TABLE 2

Main clinical characteristics of the study population

| Clinical Parameter | Controls n=50 | Colorectal Cancer Baseline n=50 | Colorectal Cancer Follow up n=23 | Ulcerative Colitis n=39 |
|---|---|---|---|---|
| Median age Years (range) | 51 (19-85) | 71 (32-90) | 70 (32-83) | 32 (22-62 |
| Gender (% females) | 8% | 48% | 43.5% | 58.3% |
| Tumor stage | | | | |
| I | - | 7 | 5 | - |
| II | - | 27 | 13 | - |
| III | - | 10 | 3 | - |
| IV | - | 3 | 2 | - |
| N/A | - | 3 | - | - |

### Statistical Analyses

One-way analysis of variance (ANOVA) adjusted for multiple comparisons with Kruskal-Wallis test were used to compare serum levels of PRO-C16 in controls, CRC patients at baseline, and UC patients. Wilcoxon test was used to compare CRC patients at baseline and at follow up. The odds ratio and positive predictive value were generated from a specific cutoff value (1.0 ng/mL), obtained from a receiver operating characteristics (ROC) curve, and analyzed using Fisher’s exact probability test and chi-square test. A p-value of p<0.05 was considered statistical significant. Statistically significant differences are marked by asterisks in figures and explained in the figure legends. Prism 7 software (Graphpad v7.01) was used for all statistical analyses.

### Specificity of the PRO-C16 Assay

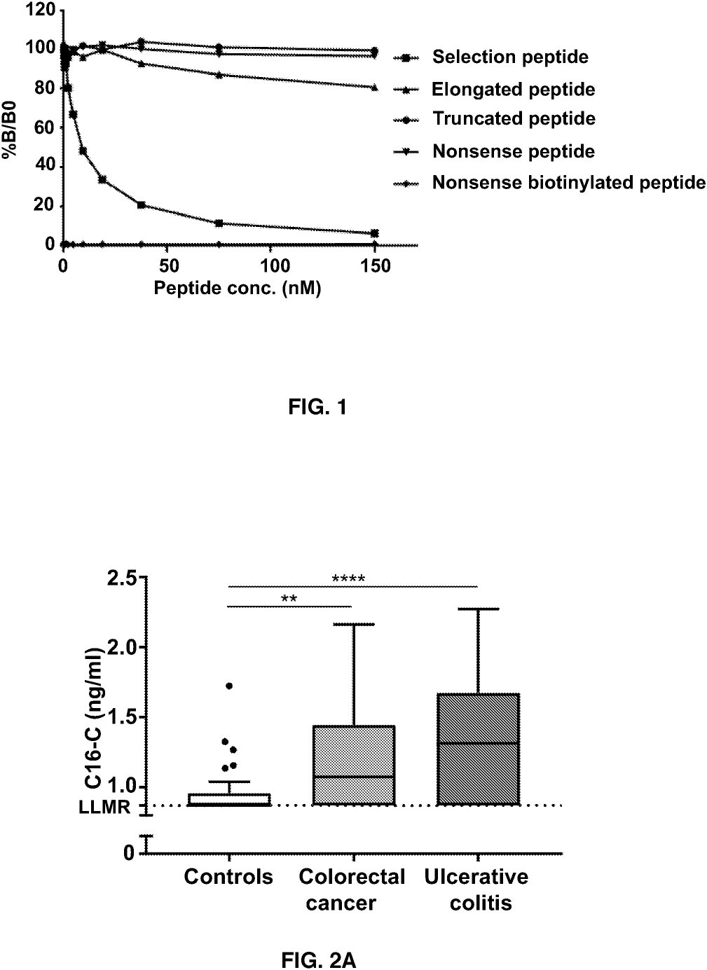

The specificity of the newly developed PRO-C16 ELISA was evaluated by investigating the inhibitory effect of different peptides. The selection peptide (PMKTMKGPFG; SEQ ID NO: 1) inhibited the signal to 6% while only a minor inhibition was detected using an elongated peptide (PMKTMKGPFGG; SEQ ID NO: 2), a truncated peptide (PMKTMKGPF; SEQ ID NO: 3) and a nonsense peptide (VPKDLPPDTT; SEQ ID NO: 5) and this only at the highest concentrations (FIG. 1). No reactivity was observed toward a nonsense biotinylated peptide (Biotin-VPKDLPPDTT; SEQ ID NO: 6). Altogether, this indicates that the antibody is specific towards the C-terminal of col-16.

### Technical Evaluation of the PRO-C16 Assay

Several tests were included to evaluate the overall technical performance of the PRO-C16 assay (Table 3). The measurement range was determined by calculating the LLMR and ULMR, which provided a range of 0.87-95.50 ng/ml. Intra- and inter-assay variation was 10% and 15%, respectively. Native reactivity was observed in human serum. The dilution recovery in serum was 95% observed from undiluted to a 1:4 dilution. Spiking of standard peptide in human serum resulted in a mean recovery of 99%, indicating accuracy and that sample matrix do not affect assay response. The stability of the analyte was acceptable after four freeze-thaw cycles with a 103% recovery. The analyte was also recovered after prolonged storage of human serum at 4° C. for 24 or 48 hours, resulting in a 106% and 95% recovery, respectively. Storage at 20° C. for 24 or 48 hours, resulted in a 91% and 85% recovery, respectively. No interference was detected from either low or high levels of biotin, lipids or haemoglobin.

TABLE 3

Technical validation of the PRO-C16 assay

| Technical validation step | Results |
|---|---|
| Detection range (LLMR-ULMR) | 0.87-95.50 ng/ml |
| Intra-assay variation | 10% |
| Inter-assay variation | 15% |
| Dilution recovery in serum | 95% |
| Spiking recovery in serum | 99% |
| Freeze-thaw recovery in serum | 103% |
| Analyte stability in serum 24h, 4° C./20° C. | 106% / 91% |
| Analyte stability in serum 48h, 4° C./20° C. | 95% / 85% |
| Interference Biotin, low/high | 94% / 113% |
| Interference Lipid, low/high | 137% / 118% |
| Interference Hemoglobin, low/high | 97% / 100% |

‘LLMR, Lower limit of measurement range; ULMR, Upper limit of measurement range. Percentages are reported as mean.

### Serum PRO-C16 Levels in Colorectal Cancer and Ulcerative Colitis

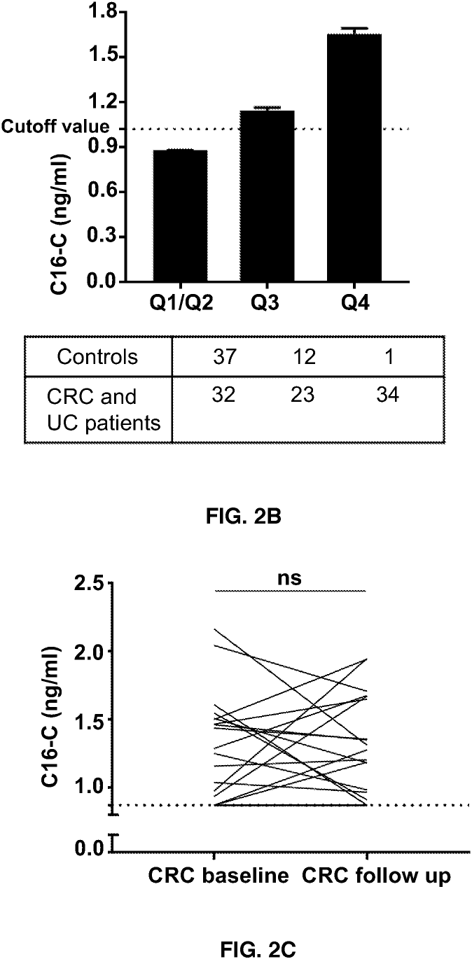

To determine the biomarker potential of col-16, PRO-C16 levels were measured in serum obtained from patients with CRC and UC and compared to healthy controls. PRO-C16 levels were significantly elevated in patients with CRC (p=0.0026) and UC (p<0.0001) compared to healthy controls (FIG. 2A). The percentage of CRC and UC cases of the total tested population increased stepwise with increasing quartile (FIG. 2B). Of the population with PRO-C16 levels in the upper quartile (Q4), 97% (34/35) were CRC or UC patients while 3% (1/35) were healthy controls. PRO-C16 was able to identify patients with CRC or UC with a positive predictive value of 0.9 and an odds ratio of 12 (95%CI=4.5-29.5, p<0.0001).

This indicates that PRO-C16 levels are able to separate patients with CRC and UC from healthy controls. Thus, measuring PRO-C16 in serum has biomarker potential in GI disorders in general.

When PRO-C16 levels were compared (paired) between the CRC patients before tumour resections (baseline) and three months after tumour resections (follow up), no difference was observed (p>0.999) (FIG. 2C). This indicates that col-16 does not originate from the primary tumour.

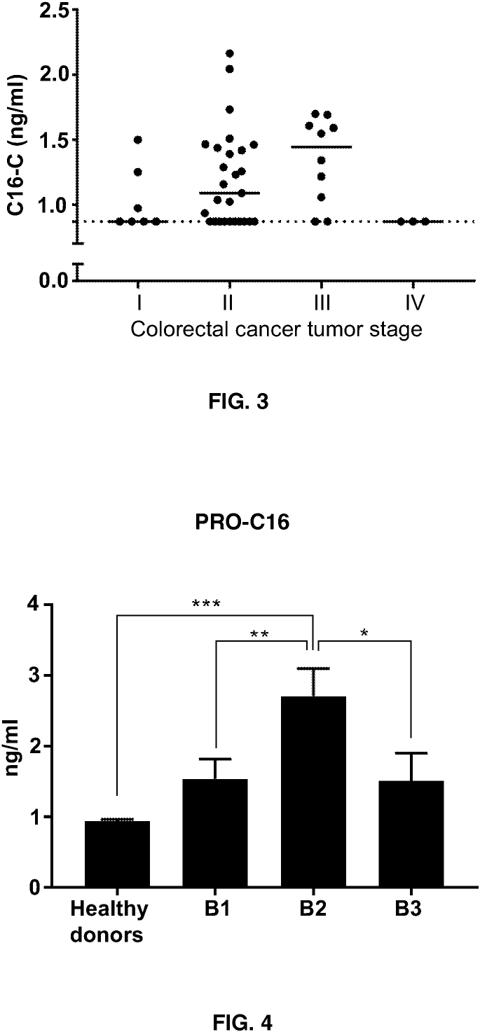

As the tumour stage is an important clinical tool in CRC, the PRO-C16 levels were divided according to tumour stage (FIG. 3). No significant difference was detected between the tumour stages. However, a trend was observed for elevated levels of PRO-C16 in stage II and III.

### Serum PRO-C16 Levels in Patients With Crohn’s Disease

Intestinal fibrosis is a common complication in inflammatory bowel disease (IBD), but is more prevalent in Crohn’s disease where it can develop into fibrostenotic strictures. Intestinal fibroblasts and myofibroblasts are the main effector cells for intestinal fibrosis development, and intestinal subepithelial myofibroblasts (ISEM) have been shown to produce significantly elevated levels of type XVI collagen in CD patients (11). As such, various subgroups of patients with Crohn’s disease (CD) were evaluated using the PRO-C16 assay:
CD patients with luminal disease (B1);
CD patients with fibrostenotic strictures disease phenotype (B2); and
CD patients with fistulizing disease phenotype (B3).
A surprisingly high (and statistically significant) level of the PRO-C16 biomarker was detected in the cohort of CD patients with fibrostenotic strictures (B2) when compared to the cohort of CD patients with luminal disease (B1), the cohort of CD patients with fistulizing disease phenotype (B3) and healthy donors (FIG. 4).

These results therefore show that the PRO-C16 assay may be used to diagnose CD patients that have or are likely to develop fibrostenotic strictures. This is a significant finding, as recent reviews of the assessment of stricturing Crohn’s disease (23, 24) note that there is a continuing need for non-invasive methods for evaluating strictures. Those reviews also note that currently there are no serological biomarkers that reliably predict the risk of developing intestinal strictures or identify early stages of fibrosis prior to clinical symptoms; none of the candidate biomarkers of intestinal fibrosis have been proven to be strictly specific for fibrostenosis. Accordingly, there is a continuing need for such specific serological biomarkers for non-invasive evaluation of CD patients with (or likely to develop) fibrostenotic strictures disease phenotype.

Thus, the statistically significant increase in levels of the PRO-C16 biomarker as identified in the B2 cohort indicates that the PRO-C16 assay may be used to reliably identify CD patients with (or likely to develop) fibrostenotic strictures. In that regard, for a sample obtained from a CD patient, a measured PRO-C16 value of at least 1.7 ng/mL (statistical cutoff value), and preferably at least 2.0 ng/mL, is considered to be indicative of a CD patient with (or likely to develop) fibrostenotic strictures.

### Conclusions

A robust competitive ELISA that enables non-invasive measurement of col-16 (PRO-C16) has been developed and validated. Using the herein described PRO-C16 assay, significantly elevated levels of PRO-C16 in serum from patients with CRC and UC were observed when compared with healthy controls. To our knowledge, this is the first study to show a link between Col-16 and UC or CRC, and we predict that there may be pathological links between Col-16 and other diseases, such as melanoma. Additionally, the PRO-C16 ELISA shows specificity for CD patients with (or likely to develop) fibrostenotic strictures disease phenotype, which is a significant improvement over the current serological biomarkers directed to this purpose.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### References

1. Kehlet SN, Sanz-Pamplona R, Brix S, Leeming DJ, Karsdal MA, Moreno V. Excessive collagen turnover products are released during colorectal cancer progression and elevated in serum from metastatic colorectal cancer patients. Sci Rep [Internet]. 2016; 6(1):30599.

2. Lawrance IC, Maxwell L, Doe W. Inflammation location, but not type, determines the increase in TGF-beta1 and IGF-1 expression and collagen deposition in IBD intestine. Inflamm Bowel Dis [Internet]. 2001; 7(1):16-26.

3. Provenzano PP, Eliceiri KW, Campbell JM, Inman DR, White JG, Keely PJ. Collagen reorganization at the tumor-stromal interface facilitates local invasion. BMC Med [Internet]. 2006; 4(1):38.

4. Egeblad M, Rasch MG, Weaver VM. Dynamic interplay between the collagen scaffold and tumor evolution. Current Opinion in Cell Biology. 2010. 22:697-706.

5. lijima J, Konno K, Itano N. Inflammatory alterations of the extracellular matrix in the tumor microenvironment. Cancers. 2011. 3:3189-205.

6. van Kempen LCL, de Visser KE, Coussens LM. Inflammation, proteases and cancer. Eur J Cancer. 2006;42(6):728-34.

7. Ravi A, Garg P, Sitaraman S V. Matrix metalloproteinases in inflammatory bowel disease: Boon or a bane? Inflammatory Bowel Diseases. 2007. 13:97-107.

8. Shimshoni E, Yablecovitch D, Baram L, Dotan I, Sagi I. ECM remodelling in IBD: innocent bystander or partner in crime? The emerging role of extracellular molecular events in sustaining intestinal inflammation. Gut [Internet]. 2015; 64(3):367-72.

9. Mortensen J Høg, Godskesen Lelbjerg, Jensen MD am, Van Haaften W Tobias, Klinge L Gabriels, Olinga P, et al. Fragments of Citrullinated and MMP-degraded Vimentin and MMP-degraded Type III Collagen Are Novel Serological Biomarkers to Differentiate Crohn’s Disease from Ulcerative Colitis. J Crohns Colitis. 2015; 9(10):863-72.

10. Rogler G. Chronic ulcerative colitis and colorectal cancer. Cancer Letters. 2014. 345:235-41.

11. Ratzinger S, Eble JA, Pasoldt A, Opolka A, Rogler G, Grifka J, et al. Collagen XVI induces formation of focal contacts on intestinal myofibroblasts isolated from the normal and inflamed intestinal tract. Matrix Biol. 2010; 29(3):177-93.

12. Grässel S, Unsöld C, Schäcke H, Bruckner-Tuderman L, Bruckner P. Collagen XVI is expressed by human dermal fibroblasts and keratinocytes and is associated with the microfibrillar apparatus in the upper papillary dermis. Matrix Biol. 1999; 18(3):309-17.

13. Kassner A, Hansen U, Miosge N, Reinhardt DP, Aigner T, Bruckner-Tuderman L, et al. Discrete integration of collagen XVI into tissue-specific collagen fibrils or beaded microfibrils. Matrix Biol. 2003; 22(2):131-43.

14. Grässel S, Bauer RJ. Collagen XVI in health and disease. Matrix Biology. 2013. 32:64-73.

15. Eble JA, Kassner A, Niland S, Mörgelin M, Grifka J, Grässel S. Collagen XVI harbors an integrin α1β1 recognition site in its C-terminal domains. J Biol Chem. 2006; 281(35):25745-56.

16. Bedal KB, Grässel S, Oefner PJ, Reinders J, Reichert TE, Bauer R. Collagen XVI induces expression of MMP9 via modulation of AP-1 transcription factors and facilitates invasion of oral squamous cell carcinoma. PLoS One. 2014; 9(1).

17. Ratzinger S, Grässel S, Dowejko A, Reichert TE, Bauer RJ. Induction of type XVI collagen expression facilitates proliferation of oral cancer cells. Matrix Biol. 2011; 30(2):118-25.

18. Bauer R, Ratzinger S, Wales L, Bosserhoff A, Senner V, Grifka J, et al. Inhibition of collagen XVI expression reduces glioma cell invasiveness. Cell Physiol Biochem. 2011; 27(3-4):217-26.

19. Senner V, Ratzinger S, Mertsch S, Grässel S, Paulus W. Collagen XVI expression is upregulated in glioblastomas and promotes tumor cell adhesion. FEBS Lett. 2008; 582(23-24):3293-300.

20. Combet C, Blanchet C, Geourjon C, Deléage G. NPS@: network protein sequence analysis. Trends Biochem Sci. 2000 Mar; 25(3):147-50.

21. Nielsen MJ, Nedergaard AF, Sun S, Veidal SS, Larsen L, Zheng Q, et al. The neo-epitope specific PRO-C3 ELISA measures true formation of type III collagen associated with liver and muscle parameters. Am J Transl Res. 2013; 5(3):303-15.

22. Gefter ML, Margulies DH, Scharff MD. A simple method for polyethylene glycol-promoted hybridization of mouse myeloma cells. Somatic Cell Genet. 1977; 3(2):231-6.

23. Giuffrida P, Pinzani M, Corazza GR, Sabatino A, Biomarkers of intestinal fibrosis – one step towards clinical trials for stricturing inflammatory bowel disease. United European Gastroenterol J. 2016 Aug; 4(4):523-530.

24. Bettenworth D, Nowacki TM, Cordes D, Buerke B, Lenze F, Assessment of stricturing Crohn’s disease: Current clinical practice and future avenues. World J Gastroenterol. 2016 Jan 21; 22(3):1008-1016.

What is claimed is:

1. A method of detecting collagen type XVI or fragments thereof in a human biofluid sample, said method comprising:
a) obtaining a biofluid sample from a human patient; and
b) contacting the biofluid sample with a monoclonal antibody specifically reactive with a C-terminus biomarker having the C-terminus amino acid sequence PMKTMKGPFG (SEQ ID NO: 1) and detecting binding between the biomarker and the antibody.

2. The method as claimed in claim 1, wherein the detection is quantitative.

3. The method as claimed in claim 1, wherein the monoclonal antibody is raised against a synthetic peptide having the amino acid sequence PMKTMKGPFG (SEQ ID NO: 1).

4. The method as claimed in claim 1, wherein the monoclonal antibody comprises at least one complementarity-determining region (CDR) that is:
CDR-L1: RSSQSIVHNNGNTYLE (SEQ ID NO: 8),
CDR-L2: KVSNRFS (SEQ ID NO: 9),
CDR-L3: FQGSHVPRT (SEQ ID NO: 10),
CDR-H1: DYYIH (SEQ ID NO: 11),
CDR-H2: WIDHDNGDTEYDPKFQG (SEQ ID NO: 12), or
CDR-H3: KGPRYGYEEDWFAY (SEQ ID NO: 13).

5. The method as claimed in claim 1, wherein the monoclonal antibody does not specifically recognise or bind a C-extended elongated version of said C-terminus amino acid sequence or a C-truncated shortened version of said C-terminus amino acid sequence.

6. The method as claimed in claim 1, wherein a measured amount of binding between the monoclonal antibody and the C-terminus biomarker of 1.0 ng/mL or greater is indicative of said human patient having or being likely to develop ulcerative colitis or colorectal cancer.

7. The method as claimed in claim 1, wherein the human patient has medical signs or symptoms indicative of colorectal cancer or ulcerative colitis.

8. The method as claimed in claim 1, wherein the human patient is a patient with Crohn’s disease, and wherein a measured amount of binding between the monoclonal antibody and the C-terminus biomarker of 1.7 ng/mL or greater is indicative of said patient having or being likely to develop fibrostenotic strictures.

9. An immunoassay method for diagnosing and/or monitoring and/or assessing the likelihood of colorectal cancer or ulcerative colitis in a patient, the method comprising contacting a biofluid sample obtained from said patient with an antibody reactive with collagen type XVI or fragments thereof, determining the amount of binding between said antibody and collagen type XVI or fragments thereof, and correlating said amount of binding with values associated with normal healthy subjects and/or values associated with known disease severity and/or values obtained from said patient at a previous time point and/or a predetermined statistical cutoff value.

10. The immunoassay method as claimed in claim 9, wherein the detection is quantitative.

11. The immunoassay method as claimed in claim 9, wherein the antibody is specifically reactive with a C-terminus biomarker having the C-terminus amino acid sequence PMKTMKGPFG (SEQ ID NO: 1).

12. The immunoassay method as claimed in claim 11, wherein the antibody is a monoclonal antibody.

13. The immunoassay method as claimed in claim 12, wherein the monoclonal antibody comprises at least one complementarity-determining region (CDR) that is:
CDR-L1: RSSQSIVHNNGNTYLE (SEQ ID NO: 8),
CDR-L2: KVSNRFS (SEQ ID NO: 9),
CDR-L3: FQGSHVPRT (SEQ ID NO: 10),
CDR-H1: DYYIH (SEQ ID NO: 11),
CDR-H2: WIDHDNGDTEYDPKFQG (SEQ ID NO: 12), or
CDR-H3: KGPRYGYEEDWFAY (SEQ ID NO: 13).

14. The immunoassay method as claimed in claim 9, wherein the antibody does not specifically recognise or bind a C-extended elongated version of said C-terminus amino acid sequence or a C-truncated shortened version of said C-terminus amino acid sequence.

15. The immunoassay method as claimed in claim 9, wherein the statistical cutoff value for the amount of binding between the monoclonal antibody and the C-terminus biomarker is at least 1.0 ng/mL.

16. The immunoassay method as claimed in claim 9, wherein the biofluid sample is blood, urine, synovial fluid, serum or plasma.

17. An immunoassay method for diagnosing the presence of fibrostenotic strictures or assessing the likelihood of development of fibrostenotic strictures in a patient with Crohn’s disease, the method comprising contacting a biofluid sample obtained from said patient with a monoclonal antibody specifically reactive with a C-terminus biomarker having the C-terminus amino acid sequence PMKTMKGPFG (SEQ ID NO: 1) and determining the amount of binding between said monoclonal antibody and said biomarker, wherein a determined amount of binding of 1.7 ng/mL or greater is indicative of the presence of or likelihood of development of fibrostenotic strictures in said patient.

18. The immunoassay method as claimed in claim 17, wherein the monoclonal antibody does not specifically recognise or bind a C-extended elongated version of said C-terminus amino acid sequence or a C-truncated shortened version of said C-terminus amino acid sequence.

19. The immunoassay method as claimed in claim 17, wherein the biofluid sample is blood, urine, synovial fluid, serum or plasma.

20. A monoclonal antibody specifically reactive with a C-terminus biomarker having the amino acid sequence PMKTMKGPFG (SEQ ID NO: 1).

21. The monoclonal antibody as claimed in claim 20, wherein the monoclonal antibody comprises at least one complementarity-determining region (CDR) that is:
CDR-L1: RSSQSIVHNNGNTYLE (SEQ ID NO: 8),
CDR-L2: KVSNRFS (SEQ ID NO: 9),
CDR-L3: FQGSHVPRT (SEQ ID NO: 10),
CDR-H1: DYYIH (SEQ ID NO: 11),
CDR-H2: WIDHDNGDTEYDPKFQG (SEQ ID NO: 12), or
CDR-H3: KGPRYGYEEDWFAY (SEQ ID NO: 13).

22. A cell line producing the monoclonal antibody of claim 21.

23. An assay kit comprising a monoclonal antibody specifically reactive with a C-terminus biomarker having the amino acid sequence PMKTMKGPFG (SEQ ID NO: 1), and at least one of:
a streptavidin coated well plate,
a biotinylated peptide Biotin-L-PMKTMKGPFG (SEQ ID NO: 4), wherein L is an optional linker,
a secondary antibody for use in a sandwich immunoassay,
a calibrator peptide comprising the sequence PMKTMKGPFG,
an antibody biotinylation kit,
an antibody HRP labeling kit,
an antibody radiolabeling kit, or
an assay visualization kit.

24. The assay kit as claimed in claim 23, wherein the monoclonal antibody is raised against a synthetic peptide having the amino acid sequence PMKTMKGPFG (SEQ ID NO: 1).

25. The assay kit as claimed in claim 23, wherein the kit is to diagnose ulcerative colitis or colorectal cancer, or to identify patients with Crohn’s disease that have or are likely to develop fibrostenotic strictures.
